(19) United States
(12) Patent Application Publication
(10) Pub. No.: US 2026/0078361 A1
(43) Pub. Date: Mar. 19, 2026

(54) DNA POLYMERASE-BASED GENOME EDITING SYSTEM AND METHOD

(71) Applicants: INSTITUTE OF GENETICS AND DEVELOPMENTAL BIOLOGY, CHINESE ACADEMY OF SCIENCES, Chaoyang District, Beijing (CN); BEIJING QI BIODESIGN BIOTECHNOLOGY COMPANY LIMITED, Changping District, Beijing (CN)

(72) Inventors: Caixia GAO, Chaoyang District, Beijing (CN); Qiupeng LIN, Guangzhou, Guangdong (CN); Guanwen LIU, Changping District, Beijing (CN); Kevin T. ZHAO, Changping District, Beijing (CN)

(21) Appl. No.: 19/109,407

(22) PCT Filed: Sep. 11, 2023
(86) PCT No.: PCT/CN2023/117975

§ 371 Date: Mar. 6, 2025

(30) Foreign Application Priority Data
Sep. 9, 2022 (CN) 202211104472.2

(51) Int. Cl. C12N 9/22; C12N 9/12; C12N 15/11; C12N 15/82

(52) U.S. Cl. CPC C12N 9/226; C12N 9/1252; C12N 15/11; C12N 15/8213; C12Y 207/07007; C07K 2319/00; C12N 2310/20

(57) ABSTRACT

The present invention relates to the field of genetic engineering. Specifically, the present invention relates to a genome editing system and method based on DNA polymerase. More specifically, the present invention relates to a method for site-directed introduction of a target modification into a genome by combining a DNA polymerase with a sequence-specific nuclease, and at the same time providing a DNA template sequence carrying the desired modification.

### TECHNICAL FIELD

The present invention relates to the field of genetic engineering. Specifically, the present invention relates to a genome editing system and method based on DNA polymerase. More specifically, the present invention relates to a method for site-directed introduction of a target modification into a genome by combining a DNA polymerase with a sequence-specific nuclease, and at the same time providing a DNA template sequence carrying the desired modification.

### BACKGROUND

Many important diseases and agronomic traits are caused by variations in the genome. By making site-directed changes to specific sequences of the genome, new heritable traits can be conferred to organisms, thereby providing the possibility for disease treatment and breeding improvement.

Currently, the homologous recombination pathway-mediated repair or prime editing system based on genome editing technology (such as CRISPR/Cas technology) are the two main methods to achieve precise rewriting of target site sequences. Among them, homologous recombination-mediated repair is carried out by providing a donor template with homologous arms, and thus the cells may have a certain probability of introducing any form of mutation into a specific site. However, the efficiency of this method is low. In the prime editing system, a reverse transcriptase is introduced into the genome editing system, fused with nCas9 that generates a non-target strand nick, and a pegRNA with RT template and primer binding site sequence at the 3′ end is provided. Rewriting of the genomic sequence at the target site then can be achieved.

The development of new methods or tools that can achieve precise replacement of target genome sequences in any form is of great significance to the field of genome editing and can be used for disease treatment, agronomic trait improvement, etc.

### BRIEF DESCRIPTION OF THE INVENTION

The present invention provides at least the following embodiments:

Embodiment 1. A genome editing system comprising:
i) a sequence-specific nuclease and/or an expression construct comprising a nucleotide sequence encoding said sequence-specific nuclease, a DNA polymerase and/or a DNA polymerase recruiting protein or an expression construct comprising a nucleotide sequence encoding said DNA polymerase and/or DNA polymerase recruiting protein, and a single-stranded DNA template;
ii) a fusion protein of a sequence-specific nuclease and a DNA polymerase or an expression construct comprising a nucleotide sequence encoding said fusion protein, and a single-stranded DNA template; or
iii) a fusion protein of a sequence-specific nuclease and a DNA polymerase recruiting protein or an expression construct comprising a nucleotide sequence encoding said fusion protein, and a single-stranded DNA template.

Embodiment 2. The genome editing system according to Embodiment 1, wherein the sequence-specific nuclease and the DNA polymerase or DNA polymerase recruiting protein in the fusion protein are linked directly or through a linker.

Embodiment 3. The genome editing system according to Embodiment 1, wherein in i) the sequence-specific nuclease and the DNA polymerase or DNA polymerase recruiting protein are capable of forming a complex, e.g., within a cell.

Embodiment 4. The genome editing system according to Embodiment 3, wherein the sequence-specific nuclease and the DNA polymerase or DNA polymerase-recruiting protein form a protein complex via affinity tags that mediate specific binding, e.g., within a cell.

Embodiment 5. The genome editing system according to any one of Embodiments 1-4, wherein the sequence-specific nuclease is selected from CRISPR nuclease, zinc finger nuclease, and transcription activator-like effector nuclease.

Embodiment 6. The genome editing system according to any one of Embodiments 1-4, wherein the sequence-specific nuclease can specifically target (bind to) a target sequence and introduce a double-stranded break (DSB) or single-stranded nick (nick) in or near the target sequence.

Embodiment 7. The genome editing system according to Embodiment 6, wherein sequence-specific nuclease of the present invention can cause the formation of a free single strand with a 3′ end (3′ free single strand) and/or a free single strand with a 5′ end (5′ free single strand) in or near the target sequence.

Embodiment 8. The genome editing system according to any one of Embodiments 1-7, wherein the sequence-specific nuclease is a CRISPR nuclease, such as a CRISPR nickase.

Embodiment 9. The genome editing system according to Embodiment 8, wherein the CRISPR nickase is a Cas9 nickase, for example, a Cas9 nickase comprising the amino acid sequence shown in SEQ ID NO:1

Embodiment 10. The genome editing system according to Embodiment 8 or 9, wherein the genome editing system further comprises a guide RNA and/or an expression construct containing a nucleotide sequence encoding the guide RNA.

Embodiment 11. The genome editing system according to any one of Embodiments 1-10, wherein the DNA polymerase is a DNA polymerase mutant with reduced such as deleted 5′-3′ exonuclease activity relative to the corresponding wild-type DNA polymerase.

Embodiment 12. The genome editing system according to Embodiment 11, wherein the 5′-3′ exonuclease domain of the DNA polymerase is mutated such that its 5′-3′ exonuclease activity is reduced, such as deleted relative to the corresponding wild-type DNA polymerase

Embodiment 13. The genome editing system according to any one of Embodiments 1-12, wherein the DNA polymerase is DNA polymerase I, such as E. coli DNA polymerase I.

Embodiment 14. The genome editing system according to Embodiment 13, wherein the E. coli DNA polymerase I comprises the amino acid sequence set forth in SEQ ID NO:2, or the E. coli DNA polymerase I with its 5′-3′ exonuclease domain deleted comprises the amino acid sequence set forth in SEQ ID NO: 11.

Embodiment 15. The genome editing system according to any one of Embodiments 1-12, wherein the DNA polymerase is T7 DNA polymerase, for example, the T7 DNA polymerase contains the amino acid sequence set forth in SEQ ID NO:3.

Embodiment 16. The genome editing system according to any one of Embodiments 1-10, wherein the DNA polymerase recruiting protein is the Rep or RepA protein of a virus, such as a plant virus.

Embodiment 17. The genome editing system according to Embodiment 16, wherein the DNA polymerase recruiting protein is the RepA protein of wheat dwarf virus, for example, the RepA protein of wheat dwarf virus comprises the amino acid sequence shown in SEQ ID NO: 4.

Embodiment 18. The genome editing system according to any one of Embodiments 1-17, wherein the single-stranded DNA template comprises at least (1) a primer binding sequence, and (2) a template sequence.

Embodiment 19. The genome editing system according to Embodiment 18, wherein the single-stranded DNA template comprises at least (1) a primer binding sequence and (2) a template sequence in order in the 3′-5′ direction.

Embodiment 20. The genome editing system according to Embodiment 19, wherein the primer binding sequence is configured to be complementary to at least a portion of the 3′ free single strand of the genomic DNA caused by the sequence-specific nuclease, in particular, complementary to the nucleotide sequence at the 3′ end of the 3′ free single strand.

Embodiment 21. The genome editing system according to any one of Embodiments 19-20, wherein the primer binding sequence is 4-20 or more nucleotides in length.

Embodiment 22. The genome editing system according to any one of Embodiments 19-21, wherein the template sequence contains a desired modification, for example, the desired modification include substitution, deletion, and/or addition of one or more nucleotides.

Embodiment 23. The genome editing system according to Embodiment 22, wherein the template sequence is configured to correspond to the sequence downstream of the nick but include the desired modification.

Embodiment 24. The genome editing system according to any one of Embodiments 19-23, wherein the template sequence is about 1-300 or more nucleotides in length.

Embodiment 25. The genome editing system according to any one of Embodiments 19-24, wherein the single-stranded DNA template further comprises one or more (3) aptamer sequences.

Embodiment 26. The genome editing system according to Embodiment 25, wherein the one or more (3) aptamer sequences are located at the 3′ or 5′ end of the single-stranded DNA template.

Embodiment 27. The genome editing system according to any one of Embodiments 25-26, wherein the sequence-specific nuclease, DNA polymerase, DNA polymerase recruiting protein and/or fusion protein further comprises a specific binding protein of the aptamer.

Embodiment 28. The genome editing system according to Embodiment 27, wherein the aptamer-specific binding protein is located at the N-terminus or C-terminus of the sequence-specific nuclease, DNA polymerase, DNA polymerase recruiting protein, and/or fusion protein.

Embodiment 29. The genome editing system according to any one of Embodiments 25-28, wherein the aptamer is RB, for example, the RB comprises the sequence set forth in SEQ ID NO:18.

Embodiment 30. The genome editing system according to Embodiment 29, wherein aptamer-specific binding protein is virD2 protein, for example, the virD2 protein comprises the sequence set forth in SEQ ID NO:14.

Embodiment 31. A method of producing a genetically modified cell, comprising introducing the genome editing system of any one of Embodiment 1-30 into at least one cell, thereby resulting in modification of a target sequence in the genome of said at least one cell.

### BRIEF DESCRIPTION OF DRAWINGS

FIG. 1: Schematic diagram of the principle of precise editing using DNA polymerase.

FIG. 2: Using DNA polymerases to achieve precise editing at endogenous sites in plant cells.

FIG. 3: Improving the efficiency of precise editing by truncation of PolI polymerase.

FIG. 4: Using the recruitment system to improve the efficiency of precision editing.

### DETAILED DESCRIPTION OF THE INVENTION

### 1. Definition

In the present invention, unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, E. F. and Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

When the term “comprise” is used herein to describe the sequence of a protein or nucleic acid, the protein or nucleic acid may consist of the sequence or may have additional amino acids or nucleotides at one or both ends of the protein or nucleic acid, but still have the activity described in this invention. In addition, those skilled in the art know that the methionine encoded by the start codon at the N-terminus of the polypeptide will be retained under certain practical conditions (for example, when expressed in a specific expression system), but does not substantially affect the function of the polypeptide. Therefore, when describing the amino acid sequence of specific polypeptide in the specification and claims of the present application, although it may not include the methionine encoded by the start codon at the N-terminus, the sequence containing the methionine is also encompassed, correspondingly, its coding nucleotide sequence may also contain a start codon; vice versa.

“Genome” as used herein encompasses not only chromosomal DNA present in the nucleus, but also organelle DNA present in the subcellular components (e.g., mitochondria, plastids) of the cell.

As used herein, an “organism” includes any organism suitable for genome editing, preferably, a eukaryote. An example of an organism includes but is not limited to, a mammal such as human, mouse, rat, monkey, dog, pig, sheep, cattle, cat; poultry such as chicken, duck, goose; a plant, including a monocotyledonous plant or a dicotyledonous plant such as rice, corn, wheat, sorghum, barley, soybean, peanut, Arabidopsis and the like.

A “genetically modified organism” or a “genetically modified cell” means an organism or a cell which comprises an exogenous polynucleotide or comprises a modified gene or expression regulatory sequence within its genome. For example, the exogenous polynucleotide can be stably integrated into the genome of the organism or cell and inherited in successive generations. The exogenous polynucleotide may be integrated into the genome alone or as a part of a recombinant DNA construct. The modified gene or expression regulatory sequence is a gene or expression regulatory sequence comprising one or more nucleotide substitutions, deletions and additions in the genome of the organism or cell.

The term “exogenous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenosine or deoxyadenosine (for RNA or DNA, respectively), “C” for cytidine or deoxycytidine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to polymers of naturally occurring amino acids. The terms “polypeptide”, “peptide”, “amino acid sequence” and “protein” are also inclusive of modifications including, but not limited to, glycosylation, lipid attachment, sulfation, gamma-carboxylation of glutamic acid residues, hydroxylation and ADP-ribosylation.

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Although there are many methods for measuring the identity between two polynucleotides or polypeptides, the term “identity” is well known to the skilled person (Carrillo, H. & Lipman, D., SIAM J Applied Math 48:1073 (1988)).

Suitable conserved amino acid replacements in peptides or proteins are known to those skilled in the art and can generally be carried out without altering the biological activity of the resulting molecule. In general, one skilled in the art recognizes that a single amino acid replacement in a non-essential region of a polypeptide does not substantially alter biological activity (See, for example, Watson et al., Molecular Biology of the Gene, 4th Edition, 1987, The Benjamin/Cummings Pub. co., p. 224).

As used herein, an “expression construct” or “construct” refers to a vector suitable for expression of a nucleotide sequence of interest in an organism, such as a recombinant vector. “Expression” refers to the production of a functional product. For example, the expression of a nucleotide sequence may refer to transcription of a nucleotide sequence (such as transcribing to produce an mRNA or a functional RNA) and/or translation of RNA into a protein precursor or a mature protein.

“Expression construct” of the invention may be a linear nucleic acid fragment, a circular plasmid, a viral vector, or, in some embodiments, an RNA that can be translated (such as an mRNA).

“Expression construct” of the invention may comprise regulatory sequences and nucleotide sequences of interest that are derived from different sources, or regulatory sequences and nucleotide sequences of interest derived from the same source but arranged in a manner different than that normally found in nature.

“Regulatory sequence” or “regulatory element” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling the transcription of another nucleic acid fragment. In some embodiments of the present invention, the promoter is a promoter capable of controlling the transcription of a gene in a cell, whether or not it is derived from the cell. The promoter may be a constitutive promoter or a tissue-specific promoter or a developmentally regulated promoter or an inducible promoter.

“Constitutive promoter” refers to a promoter that may cause expression of a gene in most circumstances in most cell types. “Tissue-specific promoter” and “tissue-preferred promoter” are used interchangeably and refer to a promoter that is expressed predominantly but not necessarily exclusively in one tissue or organ, but that may also be expressed in one specific cell or cell type. “Developmentally regulated promoter” refers to a promoter whose activity is determined by developmental events. “Inducible promoter” selectively expresses a DNA sequence operably linked to it in response to an endogenous or exogenous stimulus (environment, hormones, or chemical signals, and so on).

As used herein, the term “operably linked” means that a regulatory element (for example but not limited to, a promoter sequence, a transcription termination sequence, and so on) is associated to a nucleic acid sequence (such as a coding sequence or an open reading frame), such that the transcription of the nucleotide sequence is controlled and regulated by the transcriptional regulatory element. Techniques for operably linking a regulatory element region to a nucleic acid molecule are known in the art.

“Introduction” of a nucleic acid molecule (e.g., plasmid, linear nucleic acid fragment, RNA, etc.) or protein into an organism means that the nucleic acid or protein is used to transform a cell of the organism such that the nucleic acid or protein functions in the cell. As used in the present invention, “transformation” includes both stable transformation and transient transformation.

“Stable transformation” refers to the introduction of an exogenous nucleotide sequence into the genome, resulting in the stable inheritance of the exogenous nucleotide sequence. Once stably transformed, the exogenous nucleotide sequence is stably integrated into the genome of the organism and any of its successive generations.

“Transient transformation” refers to the introduction of a nucleic acid molecule or protein into a cell, executing its function without the stable inheritance of an exogenous nucleotide sequence. In transient transformation, the exogenous nucleotide sequence is not integrated into the genome.

“Trait” refers to the physiological, morphological, biochemical, or physical characteristics of a cell or an organism.

“Agronomic trait” is a measurable parameter including but not limited to, leaf greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, disease resistance, cold resistance, salt tolerance, and tiller number and so on.

### 2. Genome Editing System Based on DNA Polymerase

In one aspect, the invention provides a genome editing system comprising:
i) a sequence-specific nuclease and/or an expression construct comprising a nucleotide sequence encoding said sequence-specific nuclease, a DNA polymerase and/or a DNA polymerase recruiting protein or an expression construct comprising a nucleotide sequence encoding said DNA polymerase and/or DNA polymerase recruiting protein, and a single-stranded DNA template;
ii) a fusion protein of a sequence-specific nuclease and a DNA polymerase or an expression construct comprising a nucleotide sequence encoding said fusion protein, and a single-stranded DNA template; or
iii) a fusion protein of a sequence-specific nuclease and a DNA polymerase recruiting protein or an expression construct comprising a nucleotide sequence encoding said fusion protein, and a single-stranded DNA template.

In some embodiments, the sequence-specific nuclease and the DNA polymerase or DNA polymerase recruiting protein in the fusion protein are linked directly or through a linker.

The linker described herein can be a non-functional amino acid sequence without secondary structure of 1-50 (e.g., 1, 2, 3, 4, 5, 6, 7, 8, 9, 10, 11, 12, 13, 14, 15, 16, 17, 18, 19, 20 or 20-25, 25-50), or more amino acids in length. For example, the linker may be a flexible liner, such as, GGGGS, GS, GAP, (GGGGS)×3, GGS, (GGS)×7, XTEN, 32aa flexible linker (SGGSSGGSSGSETPGTSESATPESSGGSSGGS), etc.

In some embodiments, the sequence-specific nuclease and the DNA polymerase or DNA polymerase recruiting protein are capable of forming a complex, e.g., within a cell. In some embodiments, the sequence-specific nuclease and the DNA polymerase or DNA polymerase-recruiting protein form a protein complex via affinity tags that mediate specific binding, e.g., within a cell. One ordinary skill in the art can readily design suitable affinity tags to allow two or more proteins to form a complex, for example within a cell. Suitable affinity tags include, but are not limited to, various forms of protein or polypeptide interaction. For example, inteins with self-splicing function, SunTag or MoonTag etc. based on peptide epitope antigen-antibody interaction, and signal-induced receptor-ligand protein interactions such as ABA-induced ABI-PYL1, rapamycin induced FKBP-FRB, blue light-induced CRY2-CIB1, etc.

The sequence-specific nuclease may include, but is not limited to, CRISPR nuclease, zinc finger nuclease, and transcription activator-like effector nuclease. In some embodiments, the sequence-specific nuclease can specifically target (bind to) a target sequence and introduce a double-stranded break (DSB) or single-stranded nick (nick) in or near the target sequence. The sequence-specific nuclease of the present invention can cause the formation of a free single strand with a 3′ end (3′ free single strand) and/or a free single strand with a 5′ end (5′ free single strand) in or near the target sequence.

In some preferred embodiments, the sequence-specific nuclease is a CRISPR nuclease.

In some embodiments, the CRISPR nuclease is a Cas9 nuclease, such as SpCas9 derived from S. pyogenes. An exemplary wild-type SpCas9 comprises the amino acid sequence shown in SEQ ID NO:19.

In some embodiments, the CRISPR nuclease is a CRISPR nickase. The CRISPR nickase in the fusion protein is capable of forming a nick within the target sequence on the target strand (the strand on which the target sequence is located) of the genomic DNA. In some embodiments, the CRISPR nickase is a Cas9 nickase.

In some embodiments, the Cas9 nickase is derived from SpCas9 of Streptococcus pyogenes (S. pyogenes) and comprises at least the amino acid substitution H840A relative to wild-type SpCas9. In some embodiments, the Cas9 nickase comprises the amino acid sequence shown in SEQ ID NO:1. In some embodiments, the Cas9 nickase can form a nick between the −3 position nucleotide and the −4 position nucleotide of the target sequence (where the first nucleotide at the 5′ end of the PAM sequence is the +1 position).

“CRISPR nuclease” can also be derived from Cpf1 nuclease, including Cpf1 nuclease or functional variants thereof (e.g., nickases). The Cpf1 nuclease may be a Cpf1 nuclease from different species, for example from Francisella novicida U112, Acidaminococcus sp. BV3L6 or Lachnospiraceae bacterium ND2006.

Available “CRISPR nucleases” can also be derived from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Csn2, Cas4, C2c1 (Cas12b), C2c3, C2c2, Cas12c, Cas12d (i.e. CasY), Cas12e (i.e. CasX), Cas12f (i.e. Cas14), Cas12g, Cas12h, Cas12i, Cas12j (i.e. CasΦ) and other nucleases, including, for example these nucleases or functional variants thereof such as nickases.

In some embodiments, for example in the case that a CRISPR nuclease is used, the genome editing system further comprises a guide RNA and/or an expression construct containing a nucleotide sequence encoding the guide RNA.

As used herein, “guide RNA” and “gRNA” are used interchangeably and refer to RNA that can form a complex with the CRISPR effector protein and can target the complex to the target sequence due to a certain sequence identity with the target sequence. The guide RNA targets the target sequence by base pairing with the complementary strand of the target sequence. For example, the gRNA used by Cas9 nuclease or its functional variants is usually composed of crRNA and tracrRNA molecules that are partially complementary to form a complex, wherein the crRNA contains a guide sequence having sufficient identity with the target sequence so as to hybridize with the complementary strand of the target sequence and guide the CRISPR complex (Cas9+crRNA+tracrRNA) to specifically bind to the target sequence. However, it is known in the art that single guide RNA (sgRNA) can be designed, which includes both the characteristics of crRNA and tracrRNA. The gRNA used by a Cpf1 nuclease or variant thereof is usually composed only of a mature crRNA molecule, which can also be called sgRNA. It is within the ability of those skilled in the art to design a suitable gRNA based on the CRISPR nuclease used or a variant thereof and the target sequence to be edited.

The “DNA polymerase” described herein is also called DNA-dependent DNA polymerase, which can use parental DNA as a template to catalyze the polymerization of substrate dNTP molecules to form progeny DNA.

In some embodiments, the DNA polymerase is a DNA polymerase mutant with reduced 5′-3′ exonuclease activity relative to the corresponding wild-type DNA polymerase. In some embodiments, the DNA polymerase is a DNA polymerase mutant in which 5′-3′ exonuclease activity is deleted relative to the corresponding wild-type DNA polymerase. In some embodiments, the 5′-3′ exonuclease domain of the DNA polymerase is mutated such that its 5′-3′ exonuclease activity is reduced relative to the corresponding wild-type DNA polymerase. In some embodiments, the 5′-3′ exonuclease domain of the DNA polymerase is deleted such that its 5′-3′ exonuclease activity is deleted relative to the corresponding wild-type DNA polymerase.

In some embodiments, the DNA polymerase is DNA polymerase I. In some embodiments, the DNA polymerase is E. coli DNA polymerase I. An exemplary wild-type E. coli DNA polymerase I contains the amino acid sequence set forth in SEQ ID NO:2. In some embodiments, the DNA polymerase I comprises an amino acid sequence having at least 75%, at least 80%, at least 85%, at least 90%, at least 91%, at least 92%, at least 93%, at least 94%, at least 95%, at least 96%, at least 97%, at least 98%, at least 99% or even higher amino acid sequence identity with SEQ ID NO:2. In some embodiments, the 5′-3′ exonuclease domain of the E. coli DNA polymerase I is mutated, e.g., deleted. An exemplary 5′-3′ exonuclease domain of E. coli DNA polymerase I comprises the amino acid sequence corresponding to positions 1 to 322 of SEQ ID NO:2. In some embodiments, the 5′-3′ exonuclease domain of E. coli DNA polymerase I contains one or more amino acid substitutions, additions, or deletions such that it has reduced 5′-3′ exonuclease activity, preferably no 5′-3′ exonuclease activity, relative to wild-type E. coli DNA polymerase I. In some embodiments, the 5′-3′ exonuclease domain of E. coli DNA polymerase I is completely deleted. In some embodiments, the E. coli DNA polymerase I with its 5′-3′ exonuclease domain deleted comprises the amino acid sequence set forth in SEQ ID NO: 8.

In some embodiments, the DNA polymerase is T7 DNA polymerase. An exemplary T7 DNA polymerase contains the amino acid sequence set forth in SEQ ID NO:3. In some embodiments, the T7 DNA polymerase comprises at least 75%, at least 80%, at least 85%, at least 90%, at least 91%, at least 92%, at least 93%, at least 94%, at least 95%, at least 96%, at least 97%, at least 98%, at least 99% or even higher amino acid sequence identity with SEQ ID NO: 3.

Other DNA polymerases that can be used in the present invention include, but are not limited to, DNA polymerase I, DNA polymerase II, DNA polymerase III, DNA polymerase IV, DNA polymerase V, DNA polymerase α, DNA polymerase β, DNA polymerase γ, DNA polymerase δ, DNA polymerase ε, DNA polymerase τ, DNA polymerase ζ, DNA polymerase κ, DNA polymerase η, T4 DNA polymerase, φ29 DNA polymerase, Taq DNA polymerase, Bsm DNA Polymerase, Klenow fragment, TdT, Gp90, etc.

As used herein, a “DNA polymerase recruiting protein” refers to a protein capable of recruiting the cell's DNA polymerase (e.g., through protein-protein interactions) to a specific location within the cell. Exemplary DNA polymerase recruiting proteins are, for example, the Rep or RepA proteins of viruses, such as plant viruses. Through DNA polymerase recruiting proteins, intracellular DNA polymerases can be recruited to the sequence-specific nuclease.

In some specific embodiments, the DNA polymerase recruiting protein is the RepA protein of wheat dwarf virus. An exemplary RepA protein of wheat dwarf virus comprises the amino acid sequence shown in SEQ ID NO:4.

Other “DNA polymerase recruitment protein” that can be used in the present invention include, but is not limited to, replication initiation protein Rep, DnaG, PRIM1, PRIM2, CST complex, APE1, MutSβ, etc., derived from various viruses.

In some embodiments of the present invention, the sequence-specific nuclease, DNA polymerase, DNA polymerase recruitment protein and/or fusion protein of the present invention may further comprise one or more nuclear localization sequences (NLS). Generally, the one or more NLSs in the sequence-specific nuclease, DNA polymerase, DNA polymerase recruitment protein and/or fusion protein should be of sufficient strength to drive the sequence-specific nuclease, DNA polymerase, DNA polymerase recruitment protein and/or fusion protein in the nucleus of the cell to accumulate in an amount enabling its function. In general, the strength of nuclear localization activity is determined by the number, location of NLSs in the sequence-specific nuclease, DNA polymerase, DNA polymerase recruitment protein and/or fusion protein, the specific NLS(s) used, or a combination of these factors.

In some embodiments, the single-stranded DNA template comprises at least (1) a primer binding sequence, and (2) a template sequence. In some embodiments, the single-stranded DNA template comprises at least (1) a primer binding sequence and (2) a template sequence in order in the 5′-3′ direction or the 3′-5′ direction.

In some embodiments, the primer binding sequence is configured to be complementary to at least a portion of the 3′ free single strand of genomic DNA caused by the sequence-specific nuclease (preferably perfectly paired with at least a portion of the 3′ free single strand), in particular, complementary (preferably perfectly paired) to the nucleotide sequence at the 3′ end of the 3′ free single strand.

When the 3′ free single strand of the strand binds to the primer binding sequence through base pairing, the genomic DNA 3′ free single strand can serve as a primer, the template sequence immediately adjacent to the primer binding sequence can serve as a template, and DNA chain extension is performed under the action of the DNA polymerase, whereby a DNA sequence corresponding to the template sequence is extended.

The primer binding sequence depends on the length of the free single strand formed in or near the target sequence by the sequence-specific nuclease as used, however, it should be of a minimum length to ensure specific binding. In some embodiments, the primer binding sequence can be 4-20 or more nucleotides in length, for example, 4, 5, 6, 7, 8, 9, 10, 11, 12, 13, 14, 15, 16, 17, 18, 19, 20 or more nucleotides in length.

In some embodiments, the template sequence can be any sequence. Through the above-mentioned polymerase extension, its sequence information can be integrated into the single-stranded portion of genomic DNA, and then through the DNA repair function of the cell, a double-stranded genomic DNA containing the template sequence information is formed. In some embodiments, the template sequence contains the desired modification. For example, the desired modifications include substitution, deletion, and/or addition of one or more nucleotides. For example, the modification includes one or more substitutions selected from the group consisting of: C to T substitution, C to G substitution, C to A substitution, G to T substitution, G to C substitution, G to A substitution, A to T substitution, A to G substitution, A to C substitution, T to C substitution, T to G substitution, T to A substitution; and/or includes deletion of one or more nucleotides, for example, deletion of 1 to about 100 or more, for example, 1, 2, 3, 4, 5, about 10, about 20, about 30, about 40, about 50, about 75, about 100 nucleotides; and/or includes insertion of one or more nucleotides, such as 1 to about 100 or more, such as 1, 2, 3, 4, 5, about 10, about 20, about 30, about 40, about 50, about 75, or about 100 nucleotides.

In some embodiments, the template sequence is configured to correspond to the sequence downstream of the nick (e.g., complementary to at least a portion of the sequence downstream of the nick) but includes the desired modification. The desired modification includes substitution, deletion and/or addition of one or more nucleotides. For example, the modification includes one or more substitutions selected from the group consisting of: C to T substitution, C to G substitution, C to A substitution, G to T substitution, G to C substitution, G to A substitution, A to T substitution, A to G substitution, A to C substitution, T to C substitution, T to G substitution, T to A substitution; and/or includes deletion of one or more nucleotides, for example, deletion of 1 to about 100 or more, for example, 1, 2, 3, 4, 5, about 10, about 20, about 30, about 40, about 50, about 75, about 100 nucleotides; and/or includes insertion of one or more nucleotides, such as 1 to about 100 or more, such as 1, 2, 3, 4, 5, about 10, about 20, about 30, about 40, about 50, about 75, or about 100 nucleotides.

In some embodiments, the template sequence may be about 1-300 or more nucleotides in length, for example, 1, 2, 3, 4, 5, about 10, about 20, about 30, about 40, about 50, about 75, about 100, about 125, about 150, about 175, about 200, about 225, about 250, about 275, about 300 or more nucleotides in length.

In some embodiments, the single-stranded DNA template further comprises one or more (3) aptamer sequences. The aptamer can be a DNA aptamer or an RNA aptamer or a DNA/RNA hybrid aptamer, which can specifically bind to a specific protein. In some embodiments, the sequence-specific nuclease, DNA polymerase, DNA polymerase recruiting protein and/or fusion protein further comprises a specific binding protein of the aptamer, whereby the single-stranded DNA template can be recruited to the sequence-specific nuclease, DNA polymerase, DNA polymerase recruiting protein and/or fusion protein, or complexes thereof through the aptamer-aptamer specific binding protein interaction.

In some embodiments, the one or more (3) aptamer sequences are located at the 3′ end of the single-stranded DNA template. In some embodiments, the one or more (3) aptamer sequences are located at the 5′ end of the single-stranded DNA template. In some embodiments, the aptamer-specific binding protein is located at the N-terminus of the sequence-specific nuclease, DNA polymerase, DNA polymerase recruiting protein, and/or fusion protein. In some embodiments, the aptamer-specific binding protein is located at the C-terminus of the sequence-specific nuclease, DNA polymerase, DNA polymerase recruiting protein, and/or fusion protein.

In some specific embodiments, the aptamer is MS2 and the aptamer-binding protein is MCP. In some embodiments, the MS2 comprises the sequence set forth in SEQ ID NO:20. In some embodiments, the MCP protein comprises the sequence set forth in SEQ ID NO:21. In some embodiments, the MS2 is located at the 5′ end of the single-stranded DNA template. In some embodiments, the MS2 is located at the 3′ end of the single-stranded DNA template. In some specific embodiments, the aptamer is RB and the aptamer binding protein is virD2 protein. In some embodiments, the RB comprises the sequence set forth in SEQ ID NO: 18. In some embodiments, the virD2 protein comprises the sequence set forth in SEQ ID NO: 14. In some embodiments, the RB is located at the 5′ end of the single-stranded DNA template. In some embodiments, the RB is located at the 3′ end of the single-stranded DNA template.

In order to obtain effective expression in various organisms, in some embodiments of the present invention, the nucleotide sequence encoding the sequence-specific nuclease, DNA polymerase, DNA polymerase recruiting protein, and/or fusion protein is codon-optimized for the organism whose genome is to be modified.

The codon optimization refers to a method for replacing at least one codon in the natural sequence (for example, about or more than about 1, 2, 3, 4, 5, 10, 15, 20, 25, 50 or more codons) with a codon used more frequently or most frequently in the gene of the host cell, and maintaining the natural amino acid sequence while modifying the nucleic acid sequence to enhance expression in the host cell of interest. Different species exhibit specific preferences for certain codons of specific amino acids. Codon preference (difference in codon usage between organisms) is often related to the translation efficiency of messenger RNA (mRNA), which is considered as depending on the nature of the codon being translated and the availability of the specific transfer RNA (tRNA) molecule. The advantages of the selected tRNA in the cell generally reflect the codons most frequently used for peptide synthesis. Therefore, genes may be tailored to the optimal gene expression in a given organism based on codons optimization. The codon usage tables may be easily obtained, for example, in the codon usage database (“Codon Usage Database”) available at www.kazusa.orjp/codon/, and these tables may be adjusted and applied in different ways. See Nakamura Y. et al., “Codon usage tabulated from the international DNA sequence databases: status for the year 2000”. Nucl. Acids Res., 28:292 (2000).

In some embodiments, the genome editing system is used for targeted modification of the genomic DNA sequence of a cell. In some embodiments, the cell can be from, for example, a mammal such as huma, mouse, rat, monkey, dog, pig, sheep, cattle, cat; poultry such as chicken, duck, goose; a plant, including monocot and dicot, such as rice, corn, wheat, sorghum, barley, soybean, peanut, Arabidopsis, etc. In some preferred embodiments, the cell is a plant cell.

### 3. Method for Modifying a Target Sequence in the Genome of a Cell

In another aspect, the invention provides a method of producing a genetically modified cell, comprising introducing the genome editing system of the invention into at least one cell, thereby resulting in modification of a target sequence in the genome of said at least one cell. The modification include substitution, deletion, and/or addition of one or more nucleotides. For example, the modification includes one or more substitutions selected from the group consisting of: C to T substitution, C to G substitution, C to A substitution, G to T substitution, G to C substitution, G to A substitution, A to T substitution, A to G substitution, A to C substitution, T to C substitution, T to G substitution, T to A substitution; and/or includes deletion of one or more nucleotides, for example, deletion of 1 to about 100 or more, for example, 1, 2, 3, 4, 5, about 10, about 20, about 30, about 40, about 50, about 75, about 100 nucleotides; and/or includes insertion of one or more nucleotides, such as 1 to about 100 or more, such as 1, 2, 3, 4, 5, about 10, about 20, about 30, about 40, about 50, about 75, or about 100 nucleotides.

In another aspect, the present invention also provides a method of producing a genetically modified cell, comprising introducing the genome editing system of the present invention into the cell.

In another aspect, the invention also provides a genetically modified organism comprising a genetically modified cell or progeny cell thereof produced by the method of the invention.

In the present invention, the target sequence to be modified can be located anywhere in the genome, such as within a functional gene e.g., a protein-coding gene, or can be located in a gene expression regulatory region such as a promoter region or enhancer region, thereby achieving the modification of gene function or modification of gene expression. Modification in a target sequence in the cell can be detected by T7EI, PCR/RE or sequencing methods.

In the method of the present invention, the genome editing system can be introduced into the cell through various methods well known to those skilled in the art.

Methods that can be used to introduce the genome editing system of the present invention into a cell include, but are not limited to: calcium phosphate transfection, protoplast fusion, electroporation, lipofection, microinjection, viral infection (such as baculovirus, vaccinia virus, adenovirus viruses, adeno-associated viruses, lentiviruses and other viruses), biolistics, PEG-mediated protoplast transformation, Agrobacterium-mediated transformation.

Cells that can be gene edited by the method of the present invention can be from, for example, mammals such as human, mice, rats, monkeys, dogs, pigs, sheep, cattle, cats; poultry such as chickens, ducks, and geese; plants, including monocot and dicot, such as rice, corn, wheat, sorghum, barley, soybean, peanut, Arabidopsis, etc.

In some embodiments, the method of the invention is performed in vitro. For example, the cell is an isolated cell, or a cell within an isolated tissue or organ.

In some other embodiments, the method of the present invention can also be performed in vivo. For example, the cell is a cell in an organism, and the genome editing system of the present invention can be introduced into the cell in vivo by, for example, a virus- or Agrobacterium-mediated method.

### 4. Method of Producing a Genetically Modified Plant

In another aspect, the invention provides a method of producing a genetically modified plant, comprising introducing a genome editing system of the invention into at least one plant, thereby resulting in a modification in the genome of said at least one plant. The modification include substitution, deletion, and/or addition of one or more nucleotides. For example, the modification includes one or more substitutions selected from the group consisting of: C to T substitution, C to G substitution, C to A substitution, G to T substitution, G to C substitution, G to A substitution, A to T substitution, A to G substitution, A to C substitution, T to C substitution, T to G substitution, T to A substitution; and/or includes deletion of one or more nucleotides, for example, deletion of 1 to about 100 or more, for example, 1, 2, 3, 4, 5, about 10, about 20, about 30, about 40, about 50, about 75, about 100 nucleotides; and/or includes insertion of one or more nucleotides, such as 1 to about 100 or more, such as 1, 2, 3, 4, 5, about 10, about 20, about 30, about 40, about 50, about 75, or about 100 nucleotides.

In some embodiments, the method further includes screening a plant with the desired modification from the at least one plant.

In the method of the present invention, the genome editing system can be introduced into the plant by various methods well known to those skilled in the art. Methods that can be used to introduce the genome editing system of the present invention into a plant include, but are not limited to: biolistic method, PEG-mediated protoplast transformation, Agrobacterium-mediated transformation, plant virus-mediated transformation, pollen tube pathway method and ovary injection method. Preferably, the genome editing system is introduced into the plant by transient transformation.

In the method of the present invention, the modification of the genome can be achieved by simply introducing or producing the proteins, gRNA and single-stranded DNA template in the plant cell, and the modification can be stably inherited without the need to stably transform the exogenous polynucleotides encoding the components of the editing system into the plant. This avoids the potential off-target effects of a stable (continuously generating) editing system and avoids the integration of foreign nucleotide sequences in the plant genome, thereby achieving higher biosafety.

In some preferred embodiments, the introduction is performed in the absence of selection pressure, thereby avoiding integration of exogenous nucleotide sequence into the plant genome.

In some embodiments, the introduction includes transforming the genome editing system of the invention into an isolated plant cell or tissue, and then regenerating the transformed plant cell or tissue into an intact plant. Preferably, the regeneration is performed in the absence of selection pressure, that is, without the use of any selection agent against the selection gene carried on the expression vector during tissue culture. By not using a selection agent, the plant regeneration efficiency can be increased and modified plant that do not contain foreign nucleotide sequences can be obtained.

In some other embodiments, the genome editing system of the present invention can be transformed into a specific part of an intact plant, such as leave, shoot tip, pollen tube, young ear, or hypocotyl. This is particularly suitable for the transformation of plants that are difficult to regenerate in tissue culture.

In some embodiments of the invention, in vitro expressed proteins and/or in vitro transcribed RNA molecules (e.g., the expression construct is an in vitro transcribed RNA molecule) are directly transformed into the plant. The protein and/or RNA molecules can achieve genome editing in plant cells and are subsequently degraded by the cells, avoiding the integration of exogenous nucleotide sequences in the plant genome.

Therefore, in some embodiments, plant genetic modification and plant breeding using the method of the present invention can result in a plant whose genome is free of exogenous polynucleotide integration, that is, a non-transgene (transgene-free) modified plant.

In some embodiments of the invention, said modified genomic region is associated with a plant trait, such as an agronomic trait, whereby said modification results in altered (preferably improved) traits, such as agronomic traits, in said plant relative to a wild-type plant.

In some embodiments, the method further includes the step of screening a plant for desired modification and/or desired trait, such as agronomic trait.

In some embodiments of the invention, the method further includes obtaining progeny of the genetically modified plant. Preferably, the genetically modified plant or its progeny has the desired modification and/or desired trait such as agronomic trait.

In another aspect, the present invention also provides a genetically modified plant or a progeny thereof or a part thereof, wherein said plant is obtained by the above-mentioned method of the present invention. In some embodiments, the genetically modified plant or progeny thereof or part thereof is non-transgenic. Preferably, the genetically modified plant or progeny thereof has the desired genetic modification and/or the desired trait such as agronomic trait.

In another aspect, the present invention also provides a plant breeding method, comprising crossing a first genetically modified plant obtained by the above-mentioned method of the present invention with a second plant that does not contain the modification, so that the modification is transduced into the second plant. Preferably, the first genetically modified plant has desirable trait such as agronomic trait.

### EXAMPLES

### Materials and Methods

### 1. Vector Construction

The backbone of the nCas9 (H840A)-PolI, nCas9 (H840A)-T7, and nCas9 (H840A)-RepA constructs used in cell experiments is the pJIT-163 vector. PolI and T7 are derived from Escherichia coli Pol I DNA polymerase and T7 DNA polymerase respectively, and RepA is derived from the RepA replication protein of wheat dwarf virus WDV, which has the ability to recruit polymerases. The above sequences were all optimized against both rice and wheat.

The sequences recognizing the genomic target sites were constructed into the pOsU3 vector respectively. Two rice endogenous sites were selected to construct corresponding sgRNA constructs (Table 1). The single-stranded DNA template was synthesized by GenScript (Table 1), in which the two bases at the 5′ and 3′ ends of the primers used for plant cell experiments were both thio-modified.

TABLE 1
sgRNA targeting site and single-stranded DNA template sequence

| | | Single strand DNA | |
|---|---|---|---|
| Target site | Target sequence | template sequence | type of editing |
| OsCDC48-T1 | GCTAGCTTTGACATAA | A*T*T*AATGGCATTATG | +1-6 CTCCGG del |
| | TCTCCGG | TCA*A*A*G | |
| OsCDC48-T2 | GAAGGGGTCAGCGGC | C*A*G*CGTCTGGCGCA | +5 G to T |
| | GGCGCCGG | GGCGCCGCCGCTGACC | |
| | | CC*T*T*C | |

Note:
The PAM sequence in the target site sequence is shown in bold, where * represents thio modification.

### 2. Protoplast Isolation and Transformation

The protoplasts used in the present invention were derived from rice variety Zhonghua 11.

### 2.1 Rice Seedling Culture

Rice seeds were first rinsed with 75% ethanol for 1 minute, then treated with 4% sodium hypochlorite for 30 minutes, and washed with sterile water more than 5 times, cultivated on M6 medium for 3-4 weeks at 26° C., in dark.

### 2.2 Protoplast Isolation

(1) The middle parts of rice stalk were cut into 0.5-1 mm, put into 0.6 M Mannitol solution for 10 minutes in dark, then filtered, and transferred into 50 mL enzymatic solution (filtered by a 0.45 μm membrane filter), vacuumed (pressure about 15 Kpa) for 30 minutes, and placed on a shaker (10 rpm) for enzymatic hydrolysis at room temperature for 5 hours;
(2) 30-50 mL W5 was added to dilute the enzymatic hydrolyzate, and filtered through a 75 μm nylon filter into a round-bottomed centrifuge tube (50 mL);
(3) 23° C., 250 g (rcf), centrifuged for 3 minutes, the supernatant was discarded;
(4) the cells were gently suspended with 20 mL W5 and step (3) was repeated;
(5) appropriate amount of MMG was added to suspend the cells for transformation.

### 2.3 Protoplast Transformation

(1) 10 μg of each required transformation vector was added to a 2 mL centrifuge tube, mixed well, 200 μL of protoplasts was added and mixed, 220 μL of PEG4000 solution was added and mixed, placed at room temperature in the dark for 20-30 minutes of transformation induction;
(2) 880 μL W5 was added, mixed gently by inverting, 250 g (rcf), centrifuged for 3 minutes, the supernatant was discarded;
(3) 1 mL of WI solution was added, mixed gently by inverting, transferred gently to a flow tube, and incubated in the dark at room temperature for about 40 hours.

### 3. Cell DNA Extraction and Amplicon Sequencing Analysis

### 3.1 Protoplast DNA Extraction

The protoplasts were collected in a 2 mL centrifuge tube, the protoplast DNA (˜30 μL) was extracted using the CTAB method, its concentration (30-60 ng/μL) was determined using a NanoDrop ultra-micro spectrophotometer, and stored at −20° C.

### 3.2 Amplicon Sequencing Analysis

(1) Genomic primers were used to perform PCR amplification of the protoplast DNA template. The 20 μL amplification system contained 4 μL 5× Fastpfu buffer, 1.6 μL dNTPs (2.5 mM), 0.4 μL Forward primer (10 μM), 0.4 μL Reverse primer (10 μM), 0.4 μL FastPfu polymerase (2.5 U/μL), and 2 μL DNA template (˜60 ng). Amplification conditions: pre-denaturation at 95° C. for 5 min; denaturation at 95° C. for 30 s, annealing at 50-64° C. for 30 s, extension at 72° C. for 30 s, 35 cycles; full extension at 72° C. for 5 min, and storage at 12° C.;
(2) The above amplification product was diluted 10 times, and 1 μL was used as the template for the second round of PCR amplification. The amplification primer was a sequencing primer containing barcode. 50 μL amplification system contained 10 μL 5×Fastpfu buffer, 4 μL dNTPs (2.5 mM), 1 μL Forward primer (10 μM), 1 μL Reverse primer (10 μM), 1 μL FastPfu polymerase (2.5 U/μL), and 1 μL DNA template. The amplification conditions were as above, and the number of amplification cycles was 35 cycles.
(3) The PCR products were separated by 2% agarose gel electrophoresis, and the target fragments were gel recovered using the AxyPrep DNA Gel Extraction kit. The recovered products were quantitatively analyzed using a NanoDrop ultra-micro spectrophotometer; 100 ng of the recovered products were taken and mixed, and sent to Sangon Bioengineering Co., Ltd. for amplicon sequencing library construction and amplicon sequencing analysis.
(4) After sequencing, the original data was split according to sequencing primers, using WT as a control, and the editing type and editing efficiency of the products at different gene targeting sites in three repeated experiments were compared and analyzed.
   4. Observing Cell Fluorescence with Flow Cytometry

A FACSAria III (BD Biosciences) instrument was used to analyze GFP-positive protoplasts by flow cytometry.

### Example 1. Target Site Editing in Plant Cell Lines Based on DNA Polymerase

Some genome editing systems (such as nCas9) can create a nick at the target site and release a single strand. Therefore, a single-stranded DNA template can be designed to have a sequence at its 3′ end complementary to the released single strand, thereby allowing the DNA polymerase to extend the released genomic single-stranded DNA at the nick based on the information from the single-stranded DNA template. Experimental results show that this method can introduce targeted editing into the genome at endogenous sites. In addition, by introducing single-stranded binding protein to recruit single-stranded DNA template to the vicinity of the nick, the template can be enriched in situ, thereby significantly improving the editing efficiency of this method.

In order to test whether DNA polymerase can achieve precise editing in plant cells, the inventors chose to fuse DNA polymerase with nCas9 (H840A) (SEQ ID NO: 1) and deliver it with sgRNA of the target site and a single-stranded Oligo template into cells (FIG. 1). In this example, rice protoplasts were used as materials for detection, and three constructs, nCas9 (H840A)-PolI (SEQ ID NO:5), nCas9 (H840A)-T7 (SEQ ID NO:6), nCas9 (H840A)-RepA (SEQ ID NO: 7), were constructed, respectively corresponding to nCas9 (H840A) fused with E. coli PolI DNA polymerase (SEQ ID NO: 2), T7 polymerase (SEQ ID NO: 3) and wheat dwarf virus derived RepA (SEQ ID NO: 4), a protein associated with rolling circle replication.

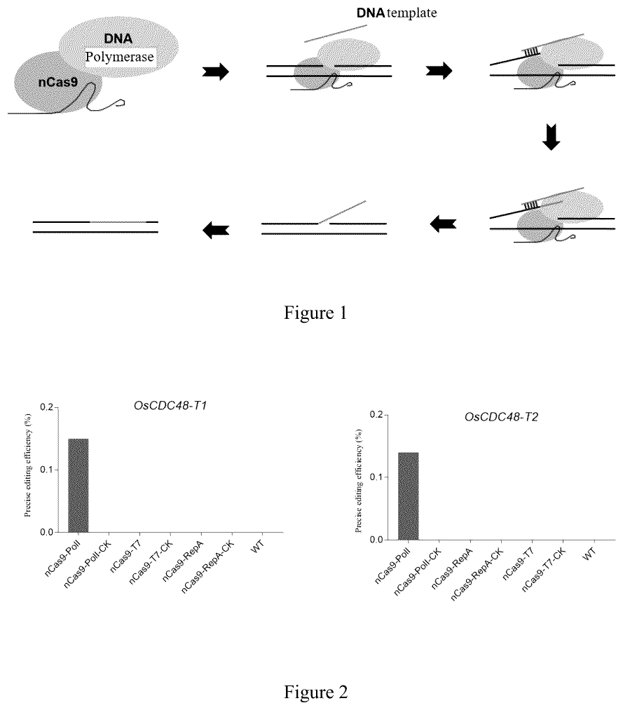

Two sgRNAs and their single-stranded DNA template sequences were designed for the OsCDC48-T1 site and OsCDC48-T2 site. Through protoplast transformation and culture, targeted deep sequencing was performed after 72 hours, and it was found that precise editing in the target site could indeed be detected. Among them, only nCas9 (H840A)-PolI showed higher activity at the endogenous site, and the efficiency at the OsCDC48-T1 site and OsCDC48-T2 site were 0.15% and 0.14% respectively (FIG. 2). In the control group (only transformed with nCas9 (H840A)-PolI and single-stranded DNA template, without transforming sgRNA), no editing events were detected, indicating that the combination of DNA polymerase genome editing system can indeed achieve precise editing in the target site. Furthermore, precise editing efficiency was only detectable using E. coli PolI DNA polymerase, while no editing events were detected using either T7 polymerase or RepA protein.

### Example 2. Optimizing E. coli PolI DNA Polymerase to Improve Editing Activity

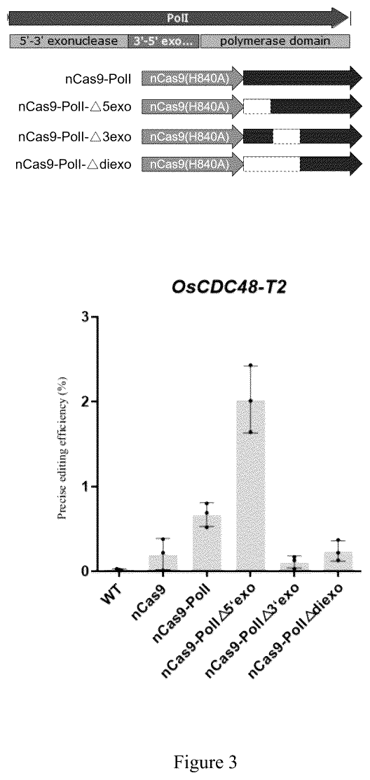

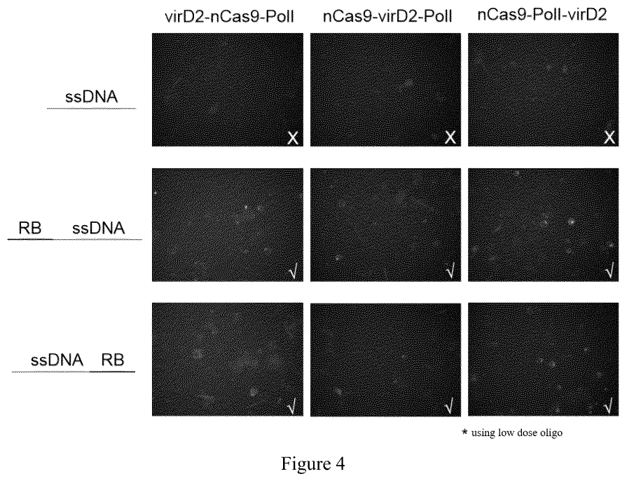

In order to further improve the efficiency of precise editing based on DNA polymerase, the structure of E. coli PolI was analyzed and found to contain three main functional domains: 5′-3′ exonuclease domain, 3′-5′ exonuclease domain, and polymerase domain. PolI was truncated, and three constructs nCas9-PolI-Δ5exo (SEQ ID NO:8), nCas9-PolI-Δ3exo (SEQ ID NO: 9), and nCas9-PolI-Δdiexo (SEQ ID NO:10), respectively corresponding to the 5′-3′ exonuclease domain deleted (SEQ ID NO:11), the 3′-5′ exonuclease domain deleted (SEQ ID NO: 12), and the two exonuclease domains deleted (SEQ ID NO:13) (FIG. 3). The activities of the above constructs and nCas9-PolI were compared through the endogenous site OsCDC48-T2. The results showed that the editing activity of the nCas9-PolI-Δ5exo construct increased significantly, and the efficiency could reach 2.03%. The above results indicate that the truncated PolI DNA polymerase with the 5′-3′ exonuclease activity removed can significantly improve the precision editing activity.

### Example 3. Recruiting Single Stranded DNA Template to Improve Precision Editing Efficiency Based on DNA Polymerase

In order to test whether recruiting the single stranded DNA template to the vicinity of the target site through recruitment methods can improve the efficiency of precise editing, we used the nCas9(H840A)-PolI construct with higher-efficiency in Example 1 for the next step of testing. virD2 protein (SEQ ID NO:14) derived from Agrobacterium was fused to the 5′ end of nCas9, between nCas9 and PolI, or to the 3′ end of PolI, respectively, to construct three f constructs: virD2-nCas9-PolI (SEQ ID NO:15), nCas9-virD2-PolI (SEQ ID NO: 16) and nCas9-PolI-virD2 (SEQ ID NO: 17). Since virD2 can bind to the RB sequence (SEQ ID NO: 18), RB sequences were designed at the 5′ end and 3′ end of the single stranded DNA template respectively to test whether the precise editing efficiency based on DNA polymerase could be improved.

Detection was performed through the GFP reporter system. If the sequence is accurately modified, the reporter system can emit green fluorescence. By transforming the constructs together with a single stranded DNA template and its corresponding reporter system vector, it was found that recruitment of single stranded DNA using virD2 allowed the reporter system emits fluorescence, indicating that the sequence of the reporter system had been accurately modified.

Sequence listing

>SEQ ID NO: 1 nCas9 (H840A)
DKKYSIGLDIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQ
SITGLYETRIDLSQLGGD
>SEQ ID NO: 2 PolI DNA Polymerase
VQIPQNPLILVDGSSYLYRAYHAFPPLTNSAGEPTGAMYGVLNMLRSLIMQYKPTHAAVVEDAKGKTFRDE
LFEHYKSHRPPMPDDLRAQIEPLHAMVKAMGLPLLAVSGVEADDVIGTLAREAEKAGRPVLISTGDKDMAQ
LVTPNITLINTMTNTILGPEEVVNKYGVPPELIIDFLALMGDSSDNIPGVPGVGEKTAQALLQGLGGLDTL
YAEPEKIAGLSFRGAKTMAAKLEQNKEVAYLSYQLATIKTDVELELTCEQLEVQQPAAEELLGLFKKYEFK
RWTADVEAGKWLQAKGAKPAAKPQETSVADEAPEVTATVISYDNYVTILDEETLKAWIAKLEKAPVFAFDT
ETDSLDNISANLVGLSFAIEPGVAAYIPVAHDYLDAPDQISRERALELLKPLLEDEKALKVGQNLKYDRGI
LANYGIELRGIAFDTMLESYILNSVAGRHDMDSLAERWLKHKTITFEEIAGKGKNQLTFNQIALEEAGRYA
AEDADVTLQLHLKMWPDLQKHKGPLNVFENIEMPLVPVLSRIERNGVKIDPKVLHNHSEELTLRLAELEKK
AHEIAGEEFNLSSTKQLQTILFEKQGIKPLKKTPGGAPSTSEEVLEELALDYPLPKVILEYRGLAKLKSTY
TDKLPLMINPKTGRVHTSYHQAVTATGRLSSTDPNLQNIPVRNEEGRRIRQAFIAPEDYVIVSADYSQIEL
RIMAHLSRDKGLLTAFAEGKDIHRATAAEVFGLPLETVTSEQRRSAKAINFGLIYGMSAFGLARQLNIPRK
EAQKYMDLYFERYPGVLEYMERTRAQAKEQGYVETLDGRRLYLPDIKSSNGARRAAAERAAINAPMQGTAA
DIIKRAMIAVDAWLQAEQPRVRMIMQVHDELVFEVHKDDVDAVAKQIHQLMENCTRLDVPLLVEVGSGENW
DQAH
>SEQ ID NO: 3 T7 DNA Polymerase
IVSDIEANALLESVTKFHCGVIYDYSTAEYVSYRPSDFGAYLDALEAEVARGGLIVFHNGHKYDVPALTKL
AKLQLNREFHLPRENCIDTLVLSRLIHSNLKDTDMGLLRSGKLPGKRFGSHALEAWGYRLGEMKGEYKDDF
KRMLEEQGEEYVDGMEWWNFNEEMMDYNVQDVVVTKALLEKLLSDKHYFPPEIDFTDVGYTTFWSESLEAV
DIEHRAAWLLAKQERNGFPFDTKAIEELYVELAARRSELLRKLTETFGSWYQPKGGTEMFCHPRTGKPLPK
YPRIKTPKVGGIFKKPKNKAQREGREPCELDTREYVAGAPYTPVEHVVENPSSRDHIQKKLQEAGWVPTKY
TDKGAPVVDDEVLEGVRVDDPEKQAAIDLIKEYLMIQKRIGQSAEGDKAWLRYVAEDGKIHGSVNPNGAVT
GRATHAFPNLAQIPGVRSPYGEQCRAAFGAEHHLDGITGKPWVQAGIDASGLELRCLAHFMARFDNGEYAH
EILNGDIHTKNQIAAELPTRDNAKTFIYGFLYGAGDEKIGQIVGAGKERGKELKKKFLENTPAIAALRESI
QQTLVESSQWVAGEQQVKWKRRWIKGLDGRKVHVRSPHAALNTLLQSAGALICKLWIIKTEEMLVEKGLKH
GWDGDFAYMAWVHDEIQVGCRTEEIAQVVIETAQEAMRWVGDHWNFRCLLDTEGKMGPNWAICH
>SEQ ID NO: 4 RepA
MASSSAPRFRVYSKYLFLTYPQCTLEPQYALDSLRTLLNKYEPLYIAAVRELHEDGSPHLHVLVQNKLRAS
ITNPNALNLRMDTSPESIFHPNIQAAKDCNQVRDYITKEVDSDVNTAEWGTFVAVSTPGRKDRDADMKQII
ESSSSREEFLSMVCNRFPFEWSIRLKDFEYTARHLFPDPVATYTPEFPTESLICHETIESWKNEHLYSVSL
ESYILCTSTPADQAQSDLEWMDDYSRSHRGGISPSTSAGQPEQERLPGQGL*GHTIIITV*LISQHMTSTP
SIISSTTFHSSSHPTGSASSGLSVTSRSIQNMVSERSYGVEYLASF
>SEQ ID NO: 5 nCas9 (H840A)-PolI
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNEMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPV
QIPQNPLILVDGSSYLYRAYHAFPPLTNSAGEPTGAMYGVLNMLRSLIMQYKPTHAAVVFDAKGKTERDEL
FEHYKSHRPPMPDDLRAQIEPLHAMVKAMGLPLLAVSGVEADDVIGTLAREAEKAGRPVLISTGDKDMAQL
VTPNITLINTMTNTILGPEEVVNKYGVPPELIIDFLALMGDSSDNIPGVPGVGEKTAQALLQGLGGLDTLY
AEPEKIAGLSFRGAKTMAAKLEQNKEVAYLSYQLATIKTDVELELTCEQLEVQQPAAEELLGLFKKYEFKR
WTADVEAGKWLQAKGAKPAAKPQETSVADEAPEVTATVISYDNYVTILDEETLKAWIAKLEKAPVFAFDTE
TDSLDNISANLVGLSFAIEPGVAAYIPVAHDYLDAPDQISRERALELLKPLLEDEKALKVGQNLKYDRGIL
ANYGIELRGIAFDTMLESYILNSVAGRHDMDSLAERWLKHKTITFEEIAGKGKNQLTFNQIALEEAGRYAA
EDADVTLQLHLKMWPDLQKHKGPLNVFENIEMPLVPVLSRIERNGVKIDPKVLHNHSEELTLRLAELEKKA
HEIAGEEFNLSSTKQLQTILFEKQGIKPLKKTPGGAPSTSEEVLEELALDYPLPKVILEYRGLAKLKSTYT
DKLPLMINPKTGRVHTSYHQAVTATGRLSSTDPNLQNIPVRNEEGRRIRQAFIAPEDYVIVSADYSQIELR
IMAHLSRDKGLLTAFAEGKDIHRATAAEVFGLPLETVTSEQRRSAKAINFGLIYGMSAFGLARQLNIPRKE
AQKYMDLYFERYPGVLEYMERTRAQAKEQGYVETLDGRRLYLPDIKSSNGARRAAAERAAINAPMQGTAAD
IIKRAMIAVDAWLQAEQPRVRMIMQVHDELVFEVHKDDVDAVAKQIHQLMENCTRLDVPLLVEVGSGENWD
QAHSGGSPKKKRKV
>SEQ ID NO: 6 nCas9 (H840A)-T7
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPI
VSDIEANALLESVTKFHCGVIYDYSTAEYVSYRPSDFGAYLDALEAEVARGGLIVFHNGHKYDVPALTKLA
KLQLNREFHLPRENCIDTLVLSRLIHSNLKDTDMGLLRSGKLPGKRFGSHALEAWGYRLGEMKGEYKDDFK
RMLEEQGEEYVDGMEWWNFNEEMMDYNVQDVVVTKALLEKLLSDKHYFPPEIDFTDVGYTTFWSESLEAVD
IEHRAAWLLAKQERNGFPFDTKAIEELYVELAARRSELLRKLTETFGSWYQPKGGTEMFCHPRTGKPLPKY
PRIKTPKVGGIFKKPKNKAQREGREPCELDTREYVAGAPYTPVEHVVFNPSSRDHIQKKLQEAGWVPTKYT
DKGAPVVDDEVLEGVRVDDPEKQAAIDLIKEYLMIQKRIGQSAEGDKAWLRYVAEDGKIHGSVNPNGAVTG
RATHAFPNLAQIPGVRSPYGEQCRAAFGAEHHLDGITGKPWVQAGIDASGLELRCLAHFMARFDNGEYAHE
ILNGDIHTKNQIAAELPTRDNAKTFIYGFLYGAGDEKIGQIVGAGKERGKELKKKFLENTPAIAALRESIQ
QTLVESSQWVAGEQQVKWKRRWIKGLDGRKVHVRSPHAALNTLLQSAGALICKLWIIKTEEMLVEKGLKHG
WDGDFAYMAWVHDEIQVGCRTEEIAQVVIETAQEAMRWVGDHWNFRCLLDTEGKMGPNWAICHSGGSPKKK
RKV
>SEQ ID NO: 7 nCas9 (H840A)-RepA
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPM
ASSSAPRFRVYSKYLFLTYPQCTLEPQYALDSLRTLLNKYEPLYIAAVRELHEDGSPHLHVLVQNKLRASI
TNPNALNLRMDTSPESIFHPNIQAAKDCNQVRDYITKEVDSDVNTAEWGTFVAVSTPGRKDRDADMKQIIE
SSSSREEFLSMVCNRFPFEWSIRLKDFEYTARHLFPDPVATYTPEFPTESLICHETIESWKNEHLYSVSLE
SYILCTSTPADQAQSDLEWMDDYSRSHRGGISPSTSAGQPEQERLPGQGL*GHTIIITV*LISQHMTSTPS
IISSTTFHSSSHPTGSASSGLSVTSRSIQNMVSERSYGVEYLASFSGGSPKKKRKV
>SEQ ID NO: 8 nCas9-Poll-?5exo
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLEKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPV
ISYDNYVTILDEETLKAWIAKLEKAPVFAFDTETDSLDNISANLVGLSFAIEPGVAAYIPVAHDYLDAPDQ
ISRERALELLKPLLEDEKALKVGQNLKYDRGILANYGIELRGIAFDTMLESYILNSVAGRHDMDSLAERWL
KHKTITFEEIAGKGKNQLTFNQIALEEAGRYAAEDADVTLQLHLKMWPDLQKHKGPLNVFENIEMPLVPVL
SRIERNGVKIDPKVLHNHSEELTLRLAELEKKAHEIAGEEFNLSSTKQLQTILFEKQGIKPLKKTPGGAPS
TSEEVLEELALDYPLPKVILEYRGLAKLKSTYTDKLPLMINPKTGRVHTSYHQAVTATGRLSSTDPNLQNI
PVRNEEGRRIRQAFIAPEDYVIVSADYSQIELRIMAHLSRDKGLLTAFAEGKDIHRATAAEVFGLPLETVT
SEQRRSAKAINFGLIYGMSAFGLARQLNIPRKEAQKYMDLYFERYPGVLEYMERTRAQAKEQGYVETLDGR
RLYLPDIKSSNGARRAAAERAAINAPMQGTAADIIKRAMIAVDAWLQAEQPRVRMIMQVHDELVFEVHKDD
VDAVAKQIHQLMENCTRLDVPLLVEVGSGENWDQAHSGGSPKKKRKV
>SEQ ID NO: 9 nCas9-PolI-A3exo
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSE
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPV
QIPQNPLILVDGSSYLYRAYHAFPPLTNSAGEPTGAMYGVLNMLRSLIMQYKPTHAAVVFDAKGKTERDEL
FEHYKSHRPPMPDDLRAQIEPLHAMVKAMGLPLLAVSGVEADDVIGTLAREAEKAGRPVLISTGDKDMAQL
VTPNITLINTMTNTILGPEEVVNKYGVPPELIIDFLALMGDSSDNIPGVPGVGEKTAQALLQGLGGLDTLY
AEPEKIAGLSFRGAKTMAAKLEQNKEVAYLSYQLATIKTDVELELTCEQLEVQQPAAEELLGLEKKYEFKR
WTADVEAGKWLQAKGAKPAAKPQETSVADEAPEVTATKHKGPLNVFENIEMPLVPVLSRIERNGVKIDPKV
LHNHSEELTLRLAELEKKAHEIAGEEFNLSSTKQLQTILFEKQGIKPLKKTPGGAPSTSEEVLEELALDYP
LPKVILEYRGLAKLKSTYTDKLPLMINPKTGRVHTSYHQAVTATGRLSSTDPNLQNIPVRNEEGRRIRQAF
IAPEDYVIVSADYSQIELRIMAHLSRDKGLLTAFAEGKDIHRATAAEVFGLPLETVTSEQRRSAKAINFGL
IYGMSAFGLARQLNIPRKEAQKYMDLYFERYPGVLEYMERTRAQAKEQGYVETLDGRRLYLPDIKSSNGAR
RAAAERAAINAPMQGTAADIIKRAMIAVDAWLQAEQPRVRMIMQVHDELVFEVHKDDVDAVAKQIHQLMEN
CTRLDVPLLVEVGSGENWDQAHSGGSPKKKRKV
>SEQ ID NO: 10 nCas9-PolI-Adiexo
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSE
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPK
HKGPLNVFENIEMPLVPVLSRIERNGVKIDPKVLHNHSEELTLRLAELEKKAHEIAGEEFNLSSTKQLQTI
LFEKQGIKPLKKTPGGAPSTSEEVLEELALDYPLPKVILEYRGLAKLKSTYTDKLPLMINPKTGRVHTSYH
QAVTATGRLSSTDPNLQNIPVRNEEGRRIRQAFIAPEDYVIVSADYSQIELRIMAHLSRDKGLLTAFAEGK
DIHRATAAEVFGLPLETVTSEQRRSAKAINFGLIYGMSAFGLARQLNIPRKEAQKYMDLYFERYPGVLEYM
ERTRAQAKEQGYVETLDGRRLYLPDIKSSNGARRAAAERAAINAPMQGTAADIIKRAMIAVDAWLQAEQPR
VRMIMQVHDELVFEVHKDDVDAVAKQIHQLMENCTRLDVPLLVEVGSGENWDQAHSGGSPKKKRKV
>SEQ ID NO: 11 PolI-A5exo
VISYDNYVTILDEETLKAWIAKLEKAPVFAFDTETDSLDNISANLVGLSFAIEPGVAAYIPVAHDYLDAPD
QISRERALELLKPLLEDEKALKVGQNLKYDRGILANYGIELRGIAFDTMLESYILNSVAGRHDMDSLAERW
LKHKTITFEEIAGKGKNQLTFNQIALEEAGRYAAEDADVTLQLHLKMWPDLQKHKGPLNVFENIEMPLVPV
LSRIERNGVKIDPKVLHNHSEELTLRLAELEKKAHEIAGEEFNLSSTKQLQTILFEKQGIKPLKKTPGGAP
STSEEVLEELALDYPLPKVILEYRGLAKLKSTYTDKLPLMINPKTGRVHTSYHQAVTATGRLSSTDPNLQN
IPVRNEEGRRIRQAFIAPEDYVIVSADYSQIELRIMAHLSRDKGLLTAFAEGKDIHRATAAEVFGLPLETV
TSEQRRSAKAINFGLIYGMSAFGLARQLNIPRKEAQKYMDLYFERYPGVLEYMERTRAQAKEQGYVETLDG
RRLYLPDIKSSNGARRAAAERAAINAPMQGTAADIIKRAMIAVDAWLQAEQPRVRMIMQVHDELVFEVHKD
DVDAVAKQIHQLMENCTRLDVPLLVEVGSGENWDQAH
>SEQ ID NO: 12 PolI-A3exo
VQIPQNPLILVDGSSYLYRAYHAFPPLTNSAGEPTGAMYGVLNMLRSLIMQYKPTHAAVVEDAKGKTERDE
LFEHYKSHRPPMPDDLRAQIEPLHAMVKAMGLPLLAVSGVEADDVIGTLAREAEKAGRPVLISTGDKDMAQ
LVTPNITLINTMTNTILGPEEVVNKYGVPPELIIDFLALMGDSSDNIPGVPGVGEKTAQALLQGLGGLDTL
YAEPEKIAGLSFRGAKTMAAKLEQNKEVAYLSYQLATIKTDVELELTCEQLEVQQPAAEELLGLFKKYEFK
RWTADVEAGKWLQAKGAKPAAKPQETSVADEAPEVTATKHKGPLNVFENIEMPLVPVLSRIERNGVKIDPK
VLHNHSEELTLRLAELEKKAHEIAGEEFNLSSTKQLQTILFEKQGIKPLKKTPGGAPSTSEEVLEELALDY
PLPKVILEYRGLAKLKSTYTDKLPLMINPKTGRVHTSYHQAVTATGRLSSTDPNLQNIPVRNEEGRRIRQA
FIAPEDYVIVSADYSQIELRIMAHLSRDKGLLTAFAEGKDIHRATAAEVFGLPLETVTSEQRRSAKAINFG
LIYGMSAFGLARQLNIPRKEAQKYMDLYFERYPGVLEYMERTRAQAKEQGYVETLDGRRLYLPDIKSSNGA
RRAAAERAAINAPMQGTAADIIKRAMIAVDAWLQAEQPRVRMIMQVHDELVFEVHKDDVDAVAKQIHQLME
NCTRLDVPLLVEVGSGENWDQAH
>SEQ ID NO: 13 PolI-Adiexo
KHKGPLNVFENIEMPLVPVLSRIERNGVKIDPKVLHNHSEELTLRLAELEKKAHEIAGEEFNLSSTKQLQT
ILFEKQGIKPLKKTPGGAPSTSEEVLEELALDYPLPKVILEYRGLAKLKSTYTDKLPLMINPKTGRVHTSY
HQAVTATGRLSSTDPNLQNIPVRNEEGRRIRQAFIAPEDYVIVSADYSQIELRIMAHLSRDKGLLTAFAEG
KDIHRATAAEVFGLPLETVTSEQRRSAKAINFGLIYGMSAFGLARQLNIPRKEAQKYMDLYFERYPGVLEY
MERTRAQAKEQGYVETLDGRRLYLPDIKSSNGARRAAAERAAINAPMQGTAADIIKRAMIAVDAWLQAEQP
RVRMIMQVHDELVFEVHKDDVDAVAKQIHQLMENCTRLDVPLLVEVGSGENWDQAH
>SEQ ID NO: 14 virD2
PDRAQVIIRIVPGGGTKTLQQIINQLEYLSRKGKLELQRSARHLDIPVPPDQIRELAQSWVTEAGIYDESQ
SDDDRQQDLTTHIIVSFPAGTDQTAAYEASREWAAEMFGSGYGGGRYNYLTAYHVDRDHPHLHVVVNRREL
LGHGWLKISRRHPQLNYDGLRKKMAEISLRHGIVLDATSRAERGIAERPITYAEHRRLERMQPW
>SEQ ID NO: 15 virD2-nCas9-PolI
PDRAQVIIRIVPGGGTKTLQQIINQLEYLSRKGKLELQRSARHLDIPVPPDQIRELAQSWVTEAGIYDESQ
SDDDRQQDLTTHIIVSFPAGTDQTAAYEASREWAAEMFGSGYGGGRYNYLTAYHVDRDHPHLHVVVNRREL
LGHGWLKISRRHPQLNYDGLRKKMAEISLRHGIVLDATSRAERGIAERPITYAEHRRLERMQPWARSGSET
PGTSESATPEMPKKKRKVDKKYSIGLDIGTNSVGWAVITDEYKVPSKKFKVLGNTDRHSIKKNLIGALLED
SGETAEATRLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLRKQRTFDNGS
IPHQIHLGELHAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRNE
MQLIHDDSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTID
RKRYTSTKEVLDATLIHQSITGLYETRIDLSQLGGDEFPKKKRKVELSGGSSGGSSGSETPGTSESATPES
SGGSSGGSRPVQIPQNPLILVDGSSYLYRAYHAFPPLTNSAGEPTGAMYGVLNMLRSLIMQYKPTHAAVVE
DAKGKTFRDELFEHYKSHRPPMPDDLRAQIEPLHAMVKAMGLPLLAVSGVEADDVIGTLAREAEKAGRPVL
ISTGDKDMAQLVTPNITLINTMTNTILGPEEVVNKYGVPPELIIDFLALMGDSSDNIPGVPGVGEKTAQAL
LQGLGGLDTLYAEPEKIAGLSFRGAKTMAAKLEQNKEVAYLSYQLATIKTDVELELTCEQLEVQQPAAEEL
LGLFKKYEFKRWTADVEAGKWLQAKGAKPAAKPQETSVADEAPEVTATVISYDNYVTILDEETLKAWIAKL
EKAPVFAFDTETDSLDNISANLVGLSFAIEPGVAAYIPVAHDYLDAPDQISRERALELLKPLLEDEKALKV
GQNLKYDRGILANYGIELRGIAFDTMLESYILNSVAGRHDMDSLAERWLKHKTITFEEIAGKGKNQLTENQ
IALEEAGRYAAEDADVTLQLHLKMWPDLQKHKGPLNVFENIEMPLVPVLSRIERNGVKIDPKVLHNHSEEL
TLRLAELEKKAHEIAGEEFNLSSTKQLQTILFEKQGIKPLKKTPGGAPSTSEEVLEELALDYPLPKVILEY
RGLAKLKSTYTDKLPLMINPKTGRVHTSYHQAVTATGRLSSTDPNLQNIPVRNEEGRRIRQAFIAPEDYVI
VSADYSQIELRIMAHLSRDKGLLTAFAEGKDIHRATAAEVFGLPLETVTSEQRRSAKAINFGLIYGMSAFG
LARQLNIPRKEAQKYMDLYFERYPGVLEYMERTRAQAKEQGYVETLDGRRLYLPDIKSSNGARRAAAERAA
INAPMQGTAADIIKRAMIAVDAWLQAEQPRVRMIMQVHDELVFEVHKDDVDAVAKQIHQLMENCTRLDVPL
LVEVGSGENWDQAHSGGSPKKKRKV
>SEQ ID NO:16 nCas9-virD2-PolI
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFKSGSETPGTSESATPEARPDRAQVIIRIVPGGGTKTLQQIINQL
EYLSRKGKLELQRSARHLDIPVPPDQIRELAQSWVTEAGIYDESQSDDDRQQDLTTHIIVSFPAGTDQTAA
YEASREWAAEMFGSGYGGGRYNYLTAYHVDRDHPHLHVVVNRRELLGHGWLKISRRHPQLNYDGLRKKMAE
ISLRHGIVLDATSRAERGIAERPITYAEHRRLERMQPWFPKKKRKVELSGGSSGGSSGSETPGTSESATPE
SSGGSSGGSRPVQIPQNPLILVDGSSYLYRAYHAFPPLTNSAGEPTGAMYGVLNMLRSLIMQYKPTHAAVV
FDAKGKTFRDELFEHYKSHRPPMPDDLRAQIEPLHAMVKAMGLPLLAVSGVEADDVIGTLAREAEKAGRPV
LISTGDKDMAQLVTPNITLINTMTNTILGPEEVVNKYGVPPELIIDFLALMGDSSDNIPGVPGVGEKTAQA
LLQGLGGLDTLYAEPEKIAGLSFRGAKTMAAKLEQNKEVAYLSYQLATIKTDVELELTCEQLEVQQPAAEE
LLGLFKKYEFKRWTADVEAGKWLQAKGAKPAAKPQETSVADEAPEVTATVISYDNYVTILDEETLKAWIAK
LEKAPVFAFDTETDSLDNISANLVGLSFAIEPGVAAYIPVAHDYLDAPDQISRERALELLKPLLEDEKALK
VGQNLKYDRGILANYGIELRGIAFDTMLESYILNSVAGRHDMDSLAERWLKHKTITFEEIAGKGKNQLTEN
QIALEEAGRYAAEDADVTLQLHLKMWPDLQKHKGPLNVFENIEMPLVPVLSRIERNGVKIDPKVLHNHSEE
LTLRLAELEKKAHEIAGEEFNLSSTKQLQTILFEKQGIKPLKKTPGGAPSTSEEVLEELALDYPLPKVILE
YRGLAKLKSTYTDKLPLMINPKTGRVHTSYHQAVTATGRLSSTDPNLQNIPVRNEEGRRIRQAFIAPEDYV
IVSADYSQIELRIMAHLSRDKGLLTAFAEGKDIHRATAAEVFGLPLETVTSEQRRSAKAINFGLIYGMSAF
GLARQLNIPRKEAQKYMDLYFERYPGVLEYMERTRAQAKEQGYVETLDGRRLYLPDIKSSNGARRAAAERA
AINAPMQGTAADIIKRAMIAVDAWLQAEQPRVRMIMQVHDELVFEVHKDDVDAVAKQIHQLMENCTRLDVP
LLVEVGSGENWDQAHSGGSPKKKRKV
>SEQ ID NO: 17 nCas9-PolI-virD2
PKKKRKVDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDAIVPQSF
LKDDSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYTSTKEVL
DATLIHQSITGLYETRIDLSQLGGDEFPKKKRKVELSGGSSGGSSGSETPGTSESATPESSGGSSGGSRPV
QIPQNPLILVDGSSYLYRAYHAFPPLTNSAGEPTGAMYGVLNMLRSLIMQYKPTHAAVVFDAKGKTERDEL
FEHYKSHRPPMPDDLRAQIEPLHAMVKAMGLPLLAVSGVEADDVIGTLAREAEKAGRPVLISTGDKDMAQL
VTPNITLINTMTNTILGPEEVVNKYGVPPELIIDFLALMGDSSDNIPGVPGVGEKTAQALLQGLGGLDTLY
AEPEKIAGLSFRGAKTMAAKLEQNKEVAYLSYQLATIKTDVELELTCEQLEVQQPAAEELLGLFKKYEFKR
WTADVEAGKWLQAKGAKPAAKPQETSVADEAPEVTATVISYDNYVTILDEETLKAWIAKLEKAPVFAFDTE
TDSLDNISANLVGLSFAIEPGVAAYIPVAHDYLDAPDQISRERALELLKPLLEDEKALKVGQNLKYDRGIL
ANYGIELRGIAFDTMLESYILNSVAGRHDMDSLAERWLKHKTITFEEIAGKGKNQLTFNQIALEEAGRYAA
EDADVTLQLHLKMWPDLQKHKGPLNVFENIEMPLVPVLSRIERNGVKIDPKVLHNHSEELTLRLAELEKKA
HEIAGEEFNLSSTKQLQTILFEKQGIKPLKKTPGGAPSTSEEVLEELALDYPLPKVILEYRGLAKLKSTYT
DKLPLMINPKTGRVHTSYHQAVTATGRLSSTDPNLQNIPVRNEEGRRIRQAFIAPEDYVIVSADYSQIELR
IMAHLSRDKGLLTAFAEGKDIHRATAAEVFGLPLETVTSEQRRSAKAINFGLIYGMSAFGLARQLNIPRKE
AQKYMDLYFERYPGVLEYMERTRAQAKEQGYVETLDGRRLYLPDIKSSNGARRAAAERAAINAPMQGTAAD
IIKRAMIAVDAWLQAEQPRVRMIMQVHDELVFEVHKDDVDAVAKQIHQLMENCTRLDVPLLVEVGSGENWD
QAHSGGSPKKKRKVKSGSETPGTSESATPEARPDRAQVIIRIVPGGGTKTLQQIINQLEYLSRKGKLELQR
SARHLDIPVPPDQIRELAQSWVTEAGIYDESQSDDDRQQDLTTHIIVSFPAGTDQTAAYEASREWAAEMFG
SGYGGGRYNYLTAYHVDRDHPHLHVVVNRRELLGHGWLKISRRHPQLNYDGLRKKMAEISLRHGIVLDATS
RAERGIAERPITYAEHRRLERMQPW
>SEQ ID NO: 18 RB
GTTTACCCGCCAATATATCCTGTCA
>SEQ ID NO: 19 SpCas9
DKKYSIGLDIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDATLIHQ
SITGLYETRIDLSQLGGD
>SEQ ID NO: 20 MS2
ACATGAGGATCACCCATGT
>SEQ ID NO: 21 MCP
ASNFTQFVLVDNGGTGDVTVAPSNFANGIAEWISSNSRSQAYKVTCSVRQSSAQNRKYTIKVEVPKGAWRS
YLNMELTIPIFATNSDCELIVKAMQGLLKDGNPIPSAIAANSGIY
>SEQ ID NO: 22
GCTAGCTTTGACATAATCTCCGG
>SEQ ID NO: 23
ATTAATGGCATTATGTCAAAG
>SEQ ID NO: 24
GAAGGGGTCAGCGGCGGCGCCGG
>SEQ ID NO: 25
CAGCGTCTGGCGCAGGCGCCGCCGCTGACCCCTTC

What we claim are:

1. A genome editing system comprising:
i) a sequence-specific nuclease and/or an expression construct comprising a nucleotide sequence encoding said sequence-specific nuclease, a DNA polymerase and/or a DNA polymerase recruiting protein or an expression construct comprising a nucleotide sequence encoding said DNA polymerase and/or DNA polymerase recruiting protein, and a single-stranded DNA template;
ii) a fusion protein of a sequence-specific nuclease and a DNA polymerase or an expression construct comprising a nucleotide sequence encoding said fusion protein, and a single-stranded DNA template; or
iii) a fusion protein of a sequence-specific nuclease and a DNA polymerase recruiting protein or an expression construct comprising a nucleotide sequence encoding said fusion protein, and a single-stranded DNA template.

2. The genome editing system according to claim 1, wherein the sequence-specific nuclease and the DNA polymerase or DNA polymerase recruiting protein in the fusion protein are linked directly or through a linker.

3. The genome editing system according to claim 1, wherein in i) the sequence-specific nuclease and the DNA polymerase or DNA polymerase recruiting protein are capable of forming a complex, e.g., within a cell.

4. The genome editing system according to claim 3, wherein the sequence-specific nuclease and the DNA polymerase or DNA polymerase-recruiting protein form a protein complex via affinity tags that mediate specific binding, e.g., within a cell.

5. The genome editing system according to any one of claims 1-4, wherein the sequence-specific nuclease is selected from CRISPR nuclease, zinc finger nuclease, and transcription activator-like effector nuclease.

6. The genome editing system according to any one of claims 1-4, wherein the sequence-specific nuclease can specifically target (bind to) a target sequence and introduce a double-stranded break (DSB) or single-stranded nick (nick) in or near the target sequence.

7. The genome editing system according to claim 6, wherein sequence-specific nuclease of the present invention can cause the formation of a free single strand with a 3′ end (3′ free single strand) and/or a free single strand with a 5′ end (5′ free single strand) in or near the target sequence.

8. The genome editing system according to any one of claims 1-7, wherein the sequence-specific nuclease is a CRISPR nuclease, such as a CRISPR nickase.

9. The genome editing system according to claim 8, wherein the CRISPR nickase is a Cas9 nickase, for example, a Cas9 nickase comprising the amino acid sequence shown in SEQ ID NO:1

10. The genome editing system according to claim 8 or 9, wherein the genome editing system further comprises a guide RNA and/or an expression construct containing a nucleotide sequence encoding the guide RNA.

11. The genome editing system according to any one of claims 1-10, wherein the DNA polymerase is a DNA polymerase mutant with reduced such as deleted 5′-3′ exonuclease activity relative to the corresponding wild-type DNA polymerase.

12. The genome editing system according to claim 11, wherein the 5′-3′ exonuclease domain of the DNA polymerase is mutated such that its 5′-3′ exonuclease activity is reduced, such as deleted relative to the corresponding wild-type DNA polymerase

13. The genome editing system according to any one of claims 1-12, wherein the DNA polymerase is DNA polymerase I, such as E. coli DNA polymerase I.

14. The genome editing system according to claim 13, wherein the E. coli DNA polymerase I comprises the amino acid sequence set forth in SEQ ID NO:2, or the E. coli DNA polymerase I with its 5′-3′ exonuclease domain deleted comprises the amino acid sequence set forth in SEQ ID NO: 11.

15. The genome editing system according to any one of claims 1-12, wherein the DNA polymerase is T7 DNA polymerase, for example, the T7 DNA polymerase contains the amino acid sequence set forth in SEQ ID NO:3.

16. The genome editing system according to any one of claims 1-10, wherein the DNA polymerase recruiting protein is the Rep or RepA protein of a virus, such as a plant virus.

17. The genome editing system according to claim 16, wherein the DNA polymerase recruiting protein is the RepA protein of wheat dwarf virus, for example, the RepA protein of wheat dwarf virus comprises the amino acid sequence shown in SEQ ID NO:4.

18. The genome editing system according to any one of claims 1-17, wherein the single-stranded DNA template comprises at least (1) a primer binding sequence, and (2) a template sequence.

19. The genome editing system according to claim 18, wherein the primer binding sequence is configured to be complementary to at least a portion of the 3′ free single strand of the genomic DNA caused by the sequence-specific nuclease, in particular, complementary to the nucleotide sequence at the 3′ end of the 3′ free single strand.

20. The genome editing system according to any one of claims 18-19, wherein the primer binding sequence is 4-20 or more nucleotides in length.

21. The genome editing system according to any one of claims 19-20, wherein the template sequence contains a desired modification, for example, the desired modification include substitution, deletion, and/or addition of one or more nucleotides.

22. The genome editing system according to claim 21, wherein the template sequence is configured to correspond to the sequence downstream of the nick but include the desired modification.

23. The genome editing system according to any one of claims 19-22, wherein the template sequence is about 1-300 or more nucleotides in length.

24. The genome editing system according to any one of claims 19-23, wherein the single-stranded DNA template further comprises one or more (3) aptamer sequences.

25. The genome editing system according to claim 24, wherein the one or more (3) aptamer sequences are located at the 3′ or 5′ end of the single-stranded DNA template.

26. The genome editing system according to any one of claims 24-25, wherein the sequence-specific nuclease, DNA polymerase, DNA polymerase recruiting protein and/or fusion protein further comprises a specific binding protein of the aptamer.

27. The genome editing system according to claim 26, wherein the aptamer-specific binding protein is located at the N-terminus or C-terminus of the sequence-specific nuclease, DNA polymerase, DNA polymerase recruiting protein, and/or fusion protein.

28. The genome editing system according to any one of claims 24-27, wherein the aptamer is RB, for example, the RB comprises the sequence set forth in SEQ ID NO:18.

29. The genome editing system according to claim 28, wherein aptamer-specific binding protein is virD2 protein, for example, the virD2 protein comprises the sequence set forth in SEQ ID NO:14.

30. A method of producing a genetically modified cell, comprising introducing the genome editing system of any one of claim 1-29 into at least one cell, thereby resulting in modification of a target sequence in the genome of said at least one cell.


SEQUENCE LISTING (25 sequences)

SEQ ID NO: 1 - protein, 1367 aa, unidentified
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 927 aa, unidentified
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 3 - protein, 703 aa, unidentified
IVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 4 - protein, 328 aa, unidentified
MASSSAPRFRVYSKYLFLTYPQCTLEPQYALDSLRTLLNKYEPLYIAAVRELHEDGSPHLHVLVQNKLRASITNPNALNLRMDTSPFSIFHPNIQAAKDCNQVRDYITKEVDSDVNTAEWGTFVAVSTPGRKDRDADMKQIIESSSSREEFLSMVCNRFPFEWSIRLKDFEYTARHLFPDPVATYTPEFPTESLICHETIESWKNEHLYSVSLESYILCTSTPADQAQSDLEWMDDYSRSHRGGISPSTSAGQPEQERLPGQGLGHTIIITVLISQHMTSTPSIISSTTFHSSSHPTGSASSGLSVTSRSIQNMVSERSYGVEYLASF

SEQ ID NO: 5 - protein, 2357 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 6 - protein, 2133 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICHSGGSPKKKRKV

SEQ ID NO: 7 - protein, 1758 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPMASSSAPRFRVYSKYLFLTYPQCTLEPQYALDSLRTLLNKYEPLYIAAVRELHEDGSPHLHVLVQNKLRASITNPNALNLRMDTSPFSIFHPNIQAAKDCNQVRDYITKEVDSDVNTAEWGTFVAVSTPGRKDRDADMKQIIESSSSREEFLSMVCNRFPFEWSIRLKDFEYTARHLFPDPVATYTPEFPTESLICHETIESWKNEHLYSVSLESYILCTSTPADQAQSDLEWMDDYSRSHRGGISPSTSAGQPEQERLPGQGLGHTIIITVLISQHMTSTPSIISSTTFHSSSHPTGSASSGLSVTSRSIQNMVSERSYGVEYLASFSGGSPKKKRKV

SEQ ID NO: 8 - protein, 2035 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 9 - protein, 2163 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 10 - protein, 1841 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 11 - protein, 605 aa, unidentified
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 12 - protein, 733 aa, unidentified
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 13 - protein, 411 aa, unidentified
KHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 14 - protein, 206 aa, unidentified
PDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPW

SEQ ID NO: 15 - protein, 2581 aa, unidentified
PDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWARSGSETPGTSESATPEMPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 16 - protein, 2582 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFKSGSETPGTSESATPEARPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWFPKKKRKVELSGGSSGGSSGSETPGTSESATPESSGGSSGGSRPVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKV

SEQ ID NO: 17 - protein, 2581 aa, unidentified
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVELSGGSSGGSSGSETPGTSESATPESSGGSSGGSRPVQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAHSGGSPKKKRKVKSGSETPGTSESATPEARPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPW

SEQ ID NO: 18 - DNA, 25 bp, unidentified
  source 1..25: mol_type = other DNA
gtttacccgccaatatatcctgtca

SEQ ID NO: 19 - protein, 1367 aa, unidentified
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - DNA, 19 bp, unidentified
  source 1..19: mol_type = other DNA
acatgaggatcacccatgt

SEQ ID NO: 21 - protein, 116 aa, unidentified
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 22 - DNA, 23 bp, unidentified
  source 1..23: mol_type = other DNA
gctagctttgacataatctccgg

SEQ ID NO: 23 - DNA, 21 bp, unidentified
  source 1..21: mol_type = other DNA
attaatggcattatgtcaaag

SEQ ID NO: 24 - DNA, 23 bp, unidentified
  source 1..23: mol_type = other DNA
gaaggggtcagcggcggcgccgg

SEQ ID NO: 25 - DNA, 35 bp, unidentified
  source 1..35: mol_type = other DNA
cagcgtctggcgcaggcgccgccgctgaccccttc